(19) United States
(12) Patent Application Publication
(10) Pub. No.: US 2003/0069628 A1
(43) Pub. Date: Apr. 10, 2003

(54) Apparatus and a method for delivery of a self-expanding medical device

(76) Inventor: Jan Otto Solem, Stetten (CH)

Correspondence Address: BROWDY AND NEIMARK, P.L.L.C., 624 Ninth Street, N.W., Washington, DC, 20001, US

(21) Appl. No.: 10/234,713

(22) Filed: Sep. 5, 2002

(30) Foreign Application Priority Data
Sep. 7, 2001 (SE) 0102969-3

(51) Int. Cl. A61F 2/06

(52) U.S. Cl. 623/1.11

(57) ABSTRACT

A self-expanding device is to be inserted into a body vessel. In a contracted state, the self-expanding device has a smaller diameter than a diameter of the body vessel and it is self-expanding to a diameter at least equal to the diameter of the body vessel when the temperature of the device exceeds a transformation temperature, which is lower than the body temperature. An apparatus for the delivery of the self-expanding device into the body vessel comprises an elongate delivery means. This is flexible for introduction into the body vessel and it has a distal part for carrying said device in its contracted state. The apparatus further comprises a cooling means having a cooling surface at said distal part and being arranged to transfer heat from said device via the cooling surface towards a proximal part of the delivery means for maintaining the self-expandable device in said contracted state at a temperature below the transformation temperature of the device.

### TECHNICAL FIELD OF THE INVENTION

The present invention is related to an apparatus and a method for delivery of a self-expanding medical device.

More precisely, the self-expanding medical device, which has a contracted state and an expanded state, is intended for delivery into a body vessel. In the contracted state, it has a diameter that is smaller than the diameter of the body vessel. When expanded it will have a diameter that is at least equal to the diameter of the body vessel. The device will self-expand when its temperature exceeds a transformation temperature, which is lower than the body temperature.

### BACKGROUND OF THE INVENTION

Balloon dilatation is today the most common therapy for diseased arteries in the whole artery tree of humans; especially in the coronary arteries the method is very common. Only in the western world 1 million patients have balloon dilatation of their coronary arteries (PTCA Percutaneous Transluminal Coronary Angioplasty) every year. Subsequent of PTCA, restenosis or early occlusion due to the obligatory vessel wall damage is common.

In more recent times, reinforcement of the artery wall by means of stenting is performed in up to 80% of the treated arteries after balloon dilatation of arterio-sclerotic diseased arteries.

By far the most common used stents are stainless steel stents being placed in position by means of a balloon, thereby achieving a permanent change of diameter of the stent. Steel stents have a tendency to recoiling and loosing contact to the vessel wall. Therefore self-expanding stents, which have an inherent striving towards an expanded state, would be of advantage in many cases. Such stents may be made of metals with a memory, one such metal being Nitinol, an alloy of nickel and titanium. However, deployment of self-expanding stents is more difficult than stainless steel stents since they have to be restrained in a contracted state by means of mechanical force. Most commonly such restraining is done by compressing the device to its contracted state and forcing it into a hollow body, for instance a tube. To deploy the device the stent has to be pushed by a piston or a pusher out of the tube. Alternatively, the outer restraining tube may be retracted from over the stent while holding the stent in position by the piston. Such methods are described in EP-A 0 183 372. However, the co-axial tube and piston system makes such systems very stiff and inflexible, thereby preventing deployment of self-expanding devices in tortuous vessels and movement around bends.

U.S. Pat. No. 4,732,152 to Wallsten et al. describes the restraining material as a double sheath, made double by everting the distal end over the proximal end. For completing deployment, the outer layer is pulled in a proximal direction, whereby the double sheath is unfolded and uncovers the stent. The high friction when pulling off such sheaths is the main reason for a very limited use of the technique.

U.S. Pat. No. 6,254,628 to Wallace et al. discloses another technique, which is limited to self-expanding rolled sheet stents. These stents are imparted with resilience to urge outward expansion of the roll through unwinding or unrolling in order to bring about contact with the inner wall of a diseased artery. The retaining mechanisms comprise tear-away sheaths which are operated with zip cord, a zip-strip construction common to commercial cellophane packaging, or a peeling construction, whereby non-sliding release of the rolled sheet stent is accomplished, i.e. the stent need not be slided relative structures for releasing the stent or vice versa.

All these above-described techniques for inserting a self-expanding device into a human body present the problem that in order to release the self-expanding device in the desired position, a manipulation is needed either by pushing the device out of a catheter or by retracting or releasing a restraining device. This implies that the release of the self-expanding device could move the self-expanding device somewhat out of the desired position. Thus, there is a risk that the stent will not be placed in the desired position. Moreover, the restraining devices makes the systems bulky and stiff and not very suitable for use in tortuous vessels.

In EP 0 443 447 a catheter is disclosed for introducing self-expanding device into a body vessel. The self-expanding device is kept in a contracted state by outflow of a cooled liquid through a side hole of the catheter. This cooled liquid prevents the temperature of the device from rising above a transformation temperature. Thus, the blood will not warm up the self-expanding device. However, the cooled liquid is introduced into the blood stream in the body and therefore only suitable cooling liquids can be used, such as saline solution. Nevertheless, adding the cooling medium to the blood will alter the blood quality in negative ways, such as by haemodilution or haemolysis, and may also alter the coagulation system of the blood.

### SUMMARY OF THE INVENTION

It is an object of the present invention to overcome the above-mentioned problems, i.e. to make possible a simple insertion of a self-expanding device into a human body in a manner which securely places the device in the desired position. It is a specific object to enable insertion of a self-expanding device into a blood vessel without altering the quality of the blood.

According to the present invention, these objects are achieved by an apparatus as claimed in claim 1 or 35, and a method as claimed in claim 36, 49 or 50. Preferred embodiments of the invention are evident from the dependent claims.

Generally, the invention is intended for any self-expanding device that is to be delivered into a body vessel, said device in a contracted state thereof having a smaller diameter than a diameter of the body vessel and being self-expanding to a diameter at least equal to the diameter of the body vessel when the temperature of the device exceeds a transformation temperature, which is lower than the body temperature. In fact, the invention could be used for delivery of such a self-expanding device anywhere in the body, e.g. into hollow organs, such as the heart chambers and cavities, the airways, the gastrointestinal organs, the biliary tract, or the urogenital tract.

More precisely, an apparatus according to the invention comprises an elongate delivery means having a distal part and a proximal part, said device being arranged for carrying said device in its contracted state at said distal part to a desired position in the body vessel. The delivery means further comprises a cooling means having a cooling surface at said distal part and being arranged to transfer heat from said device via the cooling surface towards said proximal part for maintaining the self-expandable device in said contracted state at a temperature below a transformation temperature of the device.

The objects of the invention are also achieved by an apparatus for delivery of a temperature-sensitive, self-expandable device into a body vessel, said apparatus comprising an elongate delivery means having a distal part and a proximal part. The delivery means is arranged for carrying the self-expandable device in a contracted state at said distal part to a desired position in the body vessel, and the delivery means further comprises a cooling means comprising a thermoelectric element at the distal part of the delivery means and being arranged to transfer heat from said device for maintaining the self-expandable device in said non-expanded state at a temperature below a transformation temperature of the device.

A method according to the invention for delivery of a self-expanding device of the type described above into a body vessel comprises: positioning said device on a distal part of an elongate flexible delivery means; cooling the self-expanding device when positioned on the distal part of the elongate flexible delivery means by transferring heat from the device towards a proximal part of the delivery means for maintaining the self-expanding device in its contracted state; introducing the distal part of the elongate flexible delivery means with the self-expanding device to a desired position in the body vessel; and suspending or at least reducing the cooling of the self-expanding device such that the self-expanding device is allowed to self-expand to an expanded state.

The objects of the invention are also achieved by a method a method for delivery of a self-expanding device of the type described above into a body vessel, said method comprising: positioning said device on a distal part of an elongate flexible delivery means; cooling the self-expanding device when positioned on said distal part by a thermoelectric element at said distal part of the delivery means for maintaining the self-expanding device in its contracted state; introducing said distal part of delivery means with the self-expanding device to a desired position in the body vessel, and suspending or at least reducing the cooling of the self-expanding device such that the self-expanding device is allowed to self-expand to an expanded state.

The objects of the invention are further achieved by a method for controlling expansion of a self-expandable device to be located at a position in a body vessel by the use of an elongate insertion instrument. This method comprises the step of using said insertion instrument for transferring heat from the device, during introduction of the device to said position, in order to prevent an expansion of the device.

Thus, according to the invention, the self-expanding device is held in a contracted state during the introduction to a desired position in the body by means of cooling the self-expanding device to a temperature below the transformation temperature. Thus, there is no need for a mechanical restrain of the self-expanding device and therefore, no sliding movement relative the self-expanding device is needed for releasing the restrain on the device and, thus, the positioning of the self-expanding device could be accurately controlled. Further, since heat is transferred from the device to the proximal part of the delivery means, all problems arising by the introduction of a cooling liquid into the blood vessel may be avoided.

The heat transfer from the self-expanding device prevents the temperature of the self-expanding device from rising to the transformation temperature. This temperature rise prevention implies that the rate of increase of the temperature of the device due to the warm body temperature is at least slowed down and that the temperature of the device is prohibited from rising above the transformation temperature. Thus, the cooling means need not lower the temperature of the device, it need only prevent the temperature of the device from rising too high. The temperature of the self-expanding device may increase during the initial part of the introduction until it reaches a temperature held by the cooling means. Thereafter, a further temperature rise of the self-expanding device is prevented by the cooling means. The temperature rise is prevented by heat transfer from the self-expanding device.

Preferably, the step of suspending the cooling is initiated externally of said body vessel. Then, the self-expansion of the device may be controlled from the outside of the body. This implies that a surgeon introducing the self-expanding device may fully control when the self-expansion of the device is initiated. The cooling means is preferably arranged to suspend said transfer of heat, whereby said device is allowed to self-expand to an expanded state. Thus, the self-expansion may be controlled by the suspension of the heat transfer through the cooling means.

The term “body vessel” should be interpreted as any channel for transporting fluids or air in the body, such as blood vessels, airways, biliary tracts, or urogenital tracts. The term “self-expanding device” should in this context be understood as a device presenting self-expansion properties. These self-expansion properties may cause the expansion of the device during the whole or a part of the expansion. The part of the expansion caused by the self-expansion properties may constitute the beginning or the end of the expansion. A balloon may be used for triggering the self-expansion, whereafter the self-expansion properties causes the final expansion of the device. The balloon may alternatively aid the expansion of the device during the final expansion of the device. Cooling of the device counter-acts the self-expansion properties.

The cooling means may be arranged to transfer heat during said carrying of the self-expanding device to said desired position. Thus, the self-expansion of the device is prohibited during the introduction of the device to the desired position.

In an embodiment, the cooling means comprises a cooling medium for cooling said cooling surface and for providing said transfer of heat. The cooling means may be arranged to prevent said cooling medium from entering the body vessel at said distal part. For instance, the cooling surface may be a closed surface which does not let any cooling medium through the surface. Thus, no cooling medium is introduced into the blood vessel.

An apparatus according to the invention may further comprise a means for temporary warming of the device when introduced to said desired position. Also, a method according to the invention may comprise the step of warming the self-expanding device when introduced to said desired position. The means for temporary warming may be used for accelerating the temperature rise of the self-expanding device when the device has been introduced to the desired position. By warming the self-expanding device the self-expansion of the device may be triggered earlier.

The apparatus for delivery of the self-expanding device needs to be easily handled and flexible during introduction of the self-expanding device to the desired position. The flexibility of the apparatus is achieved by the fact that no mechanical restraining means are necessary if the present invention is used, i.e. using the means for suspendable cooling for keeping the self-expanding device contracted during the introduction instead of using such mechanical restraining means. Obviously, the apparatus will only need to be flexible during the introduction of the self-expanding device. The fact that the elongate delivery means is flexible should be interpreted as the delivery means being able to be bent or formed so that it may follow an extension of a vessel during introduction. However, the flexibility need not imply that the length of the elongate delivery means is variable.

Normally, it would only be necessary to keep the temperature of the self-expanding device well below the transformation temperature until the desired position is reached.

Thus, restraining means may be used for keeping the self-expanding device contracted on the distal part of the elongate delivery means before the cooling thereof by the cooling means. Such restraining is especially important for shelf storage at room temperature, keeping the self-expanding device in place. The restraining means may be used for keeping the self-expanding device contracted until the self-expanding device is being introduced into the body.

The self-expanding device may be cooled by the cooling means while still held in a contracted state by the restraining means. However, if the transformation temperature is above room temperature, it may be sufficient to start to cool the self-expanding device when it is introduced into the body.

The restraining means may also keep the device contracted before it is placed on the distal part of the elongate delivery means. In this case, the cooling of the self-expanding device may also be initiated before it is placed on the delivery means. Thus, the self-expanding device may be cooled to a low temperature before it is placed on the delivery means.

Preferably, the restraining means is detachable from the elongate delivery means. Then, the restraining means is detached before the introduction of the device into the body. This may restrain the self-expanding device without cooling before the introduction of the device into the body.

The apparatus may comprise a second cooling means for cooling the self-expanding device before the introduction of the device into the body. Thus, the same cooling means need not be used before and during the introduction. For example, a more efficient cooling means, such as a refrigerator, may be used before the introduction of the self-expanding device into the body.

Further, the step of positioning according to the inventive method may comprise the step of keeping said device contracted on the distal part of the elongate delivery means until the step of cooling the device before the introduction of the delivery means into the body vessel. The self-expanding device may be compressed for keeping it contracted.

Preferably, the elongate delivery means is a catheter. In this case, the catheter may comprise a tube termed guide wire tube for receiving a guide wire. This guide wire may guide the catheter to the desired position during the introduction of the self-expanding device. The elongate delivery means may in turn pass through another guiding catheter if this is used.

In an alternative embodiment, the cooling means comprises a solid means extending between said distal and proximal parts for providing said transfer of heat. Then, there is no need for pumping a cooling medium through the delivery means. Preferably, the solid means is of a metal material. The solid means may be a positioning wire. Then, the elongate delivery means may be implemented as a positioning wire for carrying the self-expanding device during the introduction to the desired position. Thus, the positioning wire may both provide the transfer of heat from the device and carry the device to the desired position.

The cooling means may comprise a cooling tube for delivery of a cooling medium. In this case, the step of cooling may be provided by a cooling medium. Further, the cooling tube may be arranged in the catheter for delivery of the cooling medium to the distal part of the catheter. In this case, the step of cooling further comprises the step of delivering the cooling medium to the distal part of the catheter via a tube therein.

The catheter may further have a another tube termed draining tube for draining the cooling medium from the distal part of the catheter. A source of the cooling medium may be coupled to the cooling and draining tubes at a proximal end of the catheter. According to one embodiment, the cooling may be provided by a cooling medium delivered to the distal part of the catheter via a cooling tube therein, and drained from the distal part of the catheter via a draining tube therein.

The catheter may also comprise a balloon near its distal end or, preferably, positioned on said distal part of the catheter. Thus, an inflation of the balloon may initiate or finish the expansion of the self-expanding device.

In a preferred embodiment, the distal part of the catheter comprises at least one pad for heat transfer from the self-expanding device to the cooling means. Thus, heat is transferred from the self-expanding device to the cooling medium via at least one pad. Each pad may make contact with the self-expanding device over a substantial portion thereof, along a line or at a point thereof.

When using a metal positioning wire as said delivery means, this positioning wire preferably is isolated except for the distal part thereof. Further, the cooling means is preferably connected to a proximal part of the positioning wire. Thus, cooling a proximal part of the positioning wire may provide the cooling. The positioning wire will transfer heat to its proximal part, whereby its distal part is cooled. As a result, the self-expanding device is cooled through heat transfer from the distal part of the positioning wire.

An example of a shape memory material is nitinol, which is an alloy composed of nickel (54-60%) and titanium. Small traces of chrome, cobalt, magnesium and iron may also be present. This alloy is an ideal material for stents used according to the present invention, because of its thermal shape memory and its superelasticity.

The alloy may exist in two different forms: martensitic (or low temperature form) and austenitic (high temperature form) in which the metal assumes its required shape. For human use in vivo the austenitic temperature, i.e. the transformation temperature, ideally is around 30° C. A reversible change of the crystalline structure to a martensitic state occurs when the metal is cooled, the metal is then flexible and may be loaded into a delivery device. By further cooling the metal will remain in its contracted state without any restraining means. On release at a body temperature of 37° C., the metal will again try to return to its original shape and become more stiff and strong.

In the austenitic form the alloy becomes superelastic when the temperature is increased, for instance from 30° C. when the austenitic form occurs, to 37° C. in the human body. In this superelastic form the metal becomes rubber-like but it still has its ability and urge to return to its original shape. The superelasticity occurs when the alloy is stressed in its austenitic form, whereby it develops a stress-induced martensitic reaction, which is unstable at the higher austenitic temperature. When stress is removed, the alloy returns to its original shape by transformation from the martensitic to the austenitic state. This shape recovery occurs not because of a temperature change but because of reduction in stress.

The present invention combines the use of medical self-expanding devices with modern cryogenic technology, which today allows decreasing temperature over distance and using small calibre tubes. Thus, today equipment has been developed to create freezing temperature in the human body along catheters as thin as a fraction of a millimetre for precise application inside the human body.

Cryosurgical probes have been developed by Tortal and al. (U.S. Pat. No. 5,833,685) combining a solid cold source and a liquid cold source in the same probe. Joye et al. (U.S. Pat. No. 5,971,979) has created a cryosurgical balloon catheter for treatment of post-angioplasty hyperplasia in blood vessels. Sguazzi (U.S. Pat. No. 4,280,499) discloses cooling a probe for thermal treatment by means of transporting a refrigerant through a capillary tube into the probe and letting it expand (vaporize) in a chamber near the probe tip. All of these cryosurgical devices are used for cooling body tissue.

However, the prior art does not disclose the use of the modern cryogenic technique for controlling the shape of a self-expanding medical device and its deployment in a body vessel by suspending the cooling thereof.

It should be understood that other techniques might be used for cooling of the self-expanding medical device during the delivery and deployment thereof in the body vessel. For example, the cooling may be provided by a thermoelectric element. Thus, the cooling means may comprise a thermoelectric element at the distal part of the elongate delivery means. Thermoelectric elements are able to create a local cooling by means of electrical current. In this embodiment, a conductor may be arranged along the elongate delivery means for supplying power to the thermoelectric element from the proximal part of the delivery means. Also, a combination of cooling through a thermoelectric element and through a cooling medium may be used.

The present invention makes use of the thermal properties of such a material as the Nitinol alloy. Nitinol self-expanding devices like stents, stent grafts, and other medical device's may be contracted and formed at room temperature from an ideal functional shape, which it should have in its intended therapeutic position, to a shape that is desired for the insertion of the device. The invention may be used for delivering any memory metal medical device, such as a stent or devices for closing openings or having a valve function. For the purpose of transportation and storage, the device may be restrained in this contracted state by means of for instance a tube or a film. When the device is about to be inserted it is cooled to a low temperature much below, e.g. about 10° C. below the austenitic form temperature. Thereby the device will keep its contracted shape without any external restraining force. Now, the film or tube used to restrain the device is removed and the stent (or any other device) is naked and cool and remains in its contracted shape without the need of any external restraining force. Thus, the temperature of the device is kept low by means of modern cryogenic technique until the device is in its correct position inside the human body and is ready for release. By turning off the cooling effect of the invented delivery apparatus, and possibly adding heat instead, the device will immediately change its status back to its austenitic form and start to return to its expanded state inside the vessel to support the vessel wall. The blood of 37° C. surrounding the device during insertion through the blood vessel will have a heating effect on the metal, but this heat is transported away continuously by the cooling means.

The distal part of the catheter may have the property of being dilatable or able to swell if the cryogenic effect is turned off or if heat is supplied instead. Thus, the dilatation or swelling of the distal part of the catheter may be initiated by the suspending of the cooling. The cooling of the self-expanding device will also cool the distal part of the catheter. Thus, a suspension of the cooling of the self-expanding device will increase the temperature of the distal part of the catheter causing the swelling. This swelling might be used as a kick-off for the stent to start to expand. This kickoff effect may also be used for stents that need a small expansion to start its spontaneous expansion; one example of such a stent would be the Biflex stent produced by Jomed NV, Holland. On the other hand the cooling means might end in a balloon instead of a pad where the refrigerant agent is evaporated and keeps the stent cold. After cooling and when the stent is about to expand, the cooling refrigerant may be changed against water or saline solution for expansion of the balloon. This type of design would permit so called direct stenting with a self-expanding stent, which until now has been unheard of. Direct stenting implies that a blood vessel could be dilated and a stent could be expanded to keep the blood vessel dilated by means of the same catheter, i.e. a catheter needs to be inserted into the body only one time. Direct stenting of vascular stenosis is becoming increasingly popular in interventional vascular therapy. The refrigerant agent may also be used to inflate or expand the balloon and also cool or even freeze the tissue in contact with the stent outside the balloon as a preparation for the release of the stent. Such freezing may prevent restenosis at the treatment site.

In another embodiment of the invention, active drugs may be delivered at the site of deployment of the medical device. Today huge efforts are made to counteract or prohibit the in-growth of cells in the vessel lumen. During the last three years a lot of hope has been on radiation of the vessel wall area (branchytherapy) after implant of a stent, intending to damage the potential proliferating cells and thereby stopping them from occupying vessel lumen. Logistical problems of radiation, the failing long time results and local vessel wall problems like development of aneurysms are factors that have put further investment and research in this direction to a standstill.

Local drug therapy of the stented areas of the arteries has shown remarkable good short time results. Many studies are now ongoing to prove the long time results of such treatment. Saline infusion via local drug delivery catheter has been done without big success. However, the most common used drugs have been stereoids, antinflammatory drugs, cytostatica (Rapamycin) and chemotherapeutic drugs (Paclitaxel). Especially the last two drugs have been promising. A good method for local application of drugs in the vessel wall has still not been detected. A direct infusion by means of a needle has been tried without success due to technical difficulties and it also results in an uneven distribution of the drug. Rapamycin and Paclitaxel may not be attached to the metal and give a steady release. Now the focus of interest is to cover the stents with sheaths containing these drugs The disadvantage of this technique is that the cover sheath will block side branches in the vessel and that the release of the drug will be momentary and also the dose of drugs released will be uncertain. Also vascular grafts and stented vascular grafts are in need of good drug coverage.

Active drug delivery by means of the present invention may be performed in the following way. On the surface of the metal parts of the stent or any other medical device, small cavities or holes are made where the active drug may be deposited. After activating the cryogenic tip of the device, the cold tip is submersed into a reservoir of fluid drug. As soon as the drug enters the cavities on the surface of the device, it will become solid or even freeze. When the stent is released inside the vessel, its temperature is increased resulting in its expansion and contact with the vessel wall tissue. As a consequence, the drug will be liquid and may now be resorbed by the tissue locally. Thus, the self-expanding device or the balloon may comprise cavities containing a drug in a solid state when cooled during delivery and transformed to a liquid state when the cooling is suspended.

The active drug may also be contained in capsules or microspheres which are deposited on the stent or on a balloon positioned inside the stent. These capsules or microspheres may adhere to the balloon when cooled during delivery. Also, the use of capsules or microspheres makes a slow release of the drug possible. Thus, the self-expanding device or the balloon may comprise a drug in capsules adhering to the self-expanding device or the balloon when cooled during delivery.

### BRIEF DESCRIPTION OF THE DRAWINGS

The invention will now be described in more detail with reference to the accompanying drawings, in which:

FIGS. 1 and. 2 schematically illustrate an example of a stent in a contracted state and an expanded state, respectively,

FIGS. 3-6 are schematic views of a first embodiment of a delivery apparatus and a method of its use according to the present invention,

FIGS. 7 and 8 illustrate modifications of the first embodiment shown in FIGS. 3-6,

FIGS. 9-12 schematically illustrate a second embodiment of a delivery apparatus and a method of its use according to the present invention,

FIGS. 13-14 schematically illustrate a third embodiment of a delivery apparatus and a method of its use according to the present invention,

FIGS. 15-17 schematically illustrate a fourth embodiment of a delivery apparatus and a method of its use according to the present invention.

### DETAILED DESCRIPTION OF THE PREFERRED EMBODIMENT

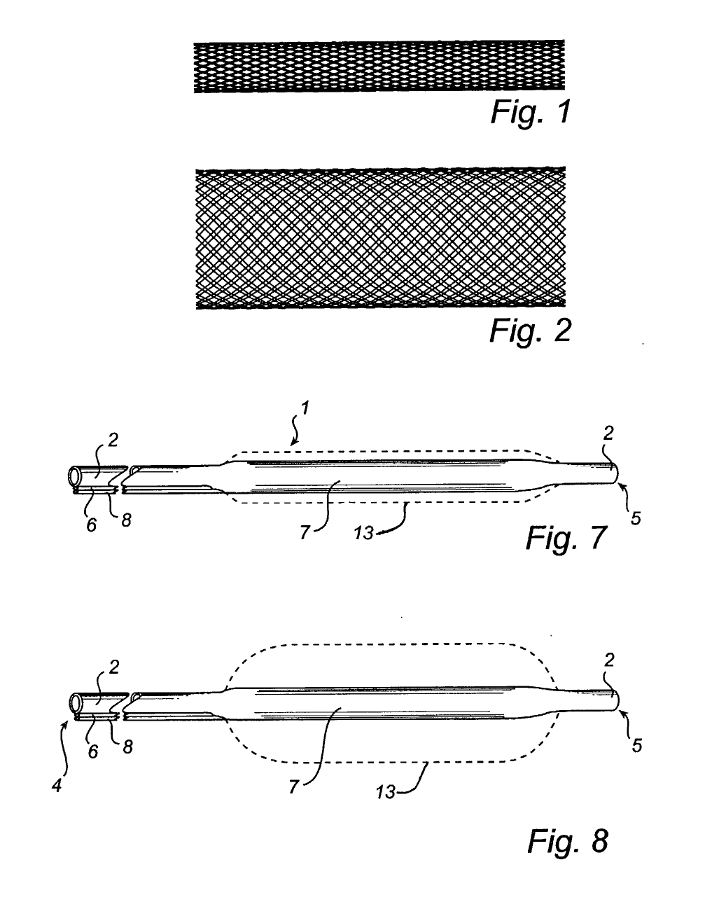

The stent in FIGS. 1 and 2 is a Nitinol stent, which is shown in its contracted state in FIG. 1 and in its expanded state in FIG. 2. The stent is of a type that will remain in its contracted state as long as its temperature is well below a transformation temperature, which should be well below the body temperature of about 37° C., e.g. 30° C. When the stent reaches a temperature above the transformation temperature, it will expand to its expanded state as a result of the temperature increase.

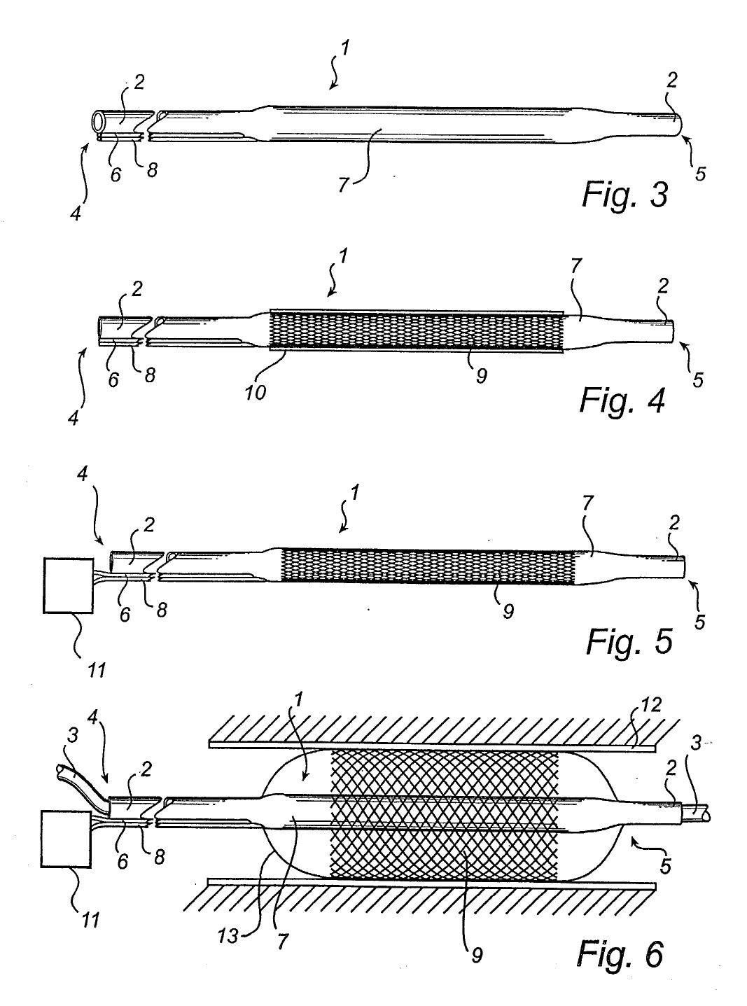

In FIGS. 3-6, a catheter 1 is shown having a tube 2 for receiving a guide wire 3, which may extend through the catheter 1 from a proximal end 4 thereof to a distal end 5 thereof. Further, the catheter 1 has a cooling tube 6 for supply of a cooling medium from the proximal end 4 of the catheter 1 to a distal part 7 thereof. This distal part 7 comprises a pad enclosing a cavity in communication with the cooling tube 6. Finally, the catheter 1 has a draining tube 8 in communication with the cavity in the distal part 7 for draining the cooling medium therefrom to the proximal end 4 of the catheter 1.

In FIG. 4, a stent 9 is positioned on the distal part 7 of the catheter 1 and is compressed to a contracted state thereon by a restraining means, e.g. a film 10. It should be understood that other types of restraining means are possible. The delivery apparatus may preferably be made and stored in the form shown in FIG. 4. When the stent is to be delivered, the cooling tube 6 is connected at its proximal end 4 to a cooling source 11 for the supply of a cooling medium to the cavity within the distal part 7. The surplus of the cooling medium is drained via the draining tube 8 back to the proximal end 4 of the catheter 1 for return to the cooling source 11 for recycling or for expelling into the atmosphere. The cooling medium could for example be cold water or a gas, such as a freon.

As soon as the temperature of the stent 9 positioned on the distal part 7 of the catheter 1 has decreased well below the transformation temperature, e.g. about 10° C. below a transformation temperature of about 30° C. or as close as possible to the freezing point, the restraining means 10 may be removed and the stent 9 will remain in its contracted state fixed on the distal part 7 of the catheter 1, as illustrated in FIG. 5.

Next, the catheter 1 is introduced into a body vessel 12 on the guide wire 3, or through a guiding catheter (not shown), to a desired site where the stent 9 is to be deployed. Here, the supply of the cooling medium from the cooling source 11 to the pad 7 is suspended or interrupted, whereby the temperature of the stent 9 will increase above the transformation temperature and the stent 9 will dilate to its expanded state and press against the inner wall of the body vessel 12 exactly at the desired position, as illustrated in FIG. 6. Finally, the catheter 1 and the guide wire 3 are withdrawn from the body vessel 12.

The cooling medium needs to have a temperature which is low enough for preventing the temperature of the stent 9 from rising above the transformation temperature. When the introduction of the stent 9 is initiated, the cooling medium may have a higher temperature than the stent 9. The temperature of the cooling medium may then be sufficient for preventing the temperature of the stent 9 from rising above the transformation temperature. As an example, the stent 9 may be cooled to a temperature of 10° C. or as close as possible to the freezing point before it is introduced into the body. This initial cooling may be accomplished by a conventional cooling means, such as a refrigerator. Then, the temperature of the cooling medium may be 10-15° C. during the introduction of the stent 9. This temperature of the cooling medium will prevent the temperature of the stent 9 from rising above the transformation temperature. When the stent 9 has been introduced to the desired position, the cooling tube may be used for supplying a warm medium to the distal part of the catheter. Thus, a medium of 42° C. may be supplied for initiating and triggering the expansion of the stent 9.

Alternatively, the stent may be of a bi-stable type that will expand by a combination of having a temperature above the transformation temperature and being triggered mechanically from its contracted state towards its expanded state. Such stents are made by Jomed NV, Holland, and sold as “Biflex” stents. In this case, the pad 7 may be made of a material that swells or expands when attaining a temperature above the transformation temperature of the stent in order to provide the mechanical triggering, as illustrated in FIG. 7. A balloon 13 positioned inside the stent 9 on the distal part 7 might also perform the mechanical triggering. By inflating the balloon 13 at a temperature above the transition temperature, the stent 9 is triggered to dilate to its expanded state, as illustrated in FIG. 8. The balloon 13 may also be used for post delivery dilatation of the treated site of the vessel or for delivery of drugs thereto. The balloon 13 may also be used for cooling, i.e. substituting or complementing the cooling by the pad and the cavity at the distal part of the elongate delivery means.

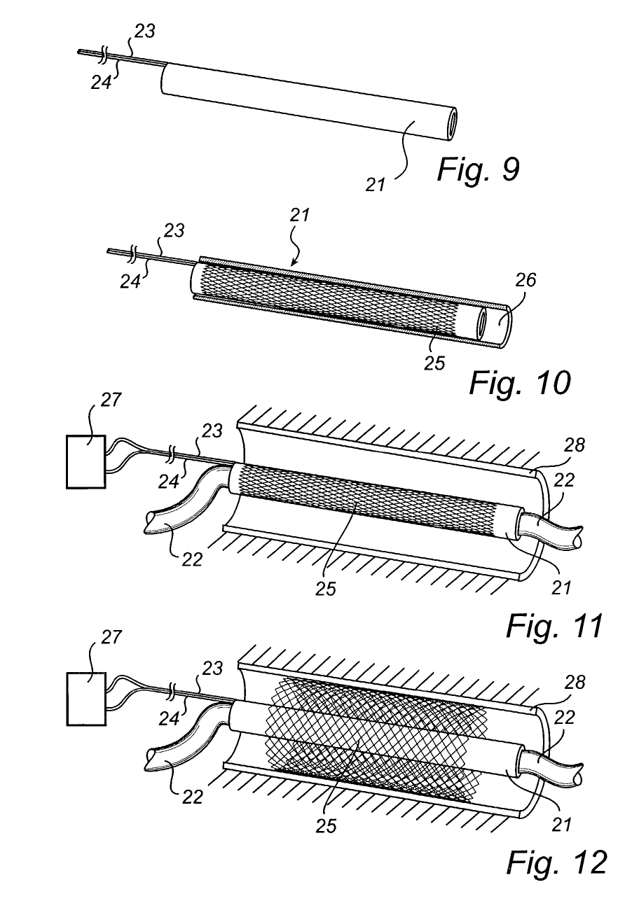

In FIGS. 9-12, the second embodiment of a delivery apparatus and a method of its use is shown as comprising a cooling pad 21 having a through hole for receiving a guide wire 22, a cooling tube 23 for supply of a cooling medium to the cooling pad 21 and a draining tube 24 for draining cooling medium from the cooling pad 21.

FIG. 10 illustrates the delivery apparatus as it may be delivered and stored before use. More precisely, a stent 25 is fixed on the cooling pad 21, i.e. in a contracted state, by means of a film or tube 26.

In FIG. 11, the cooling tube 23 has been connected to a source 27 of the cooling medium and the film 26 has been removed from the cooling pad 21. The cooling provided by the cooling medium that is being supplied to the cooling pad 21 keeps the stent 25 in its contracted state fixed on the cooling pad 21 also during the insertion thereof to a desired site in a body vessel 28.

Then the supply of the cooling medium is suspended, i.e. shut off, whereby the temperature of the stent 25 increases and exceeds the transformation temperature such that the stent 25 dilates to its expanded state where it presses against the inside wall of the body vessel 28, as illustrated in FIG. 12.

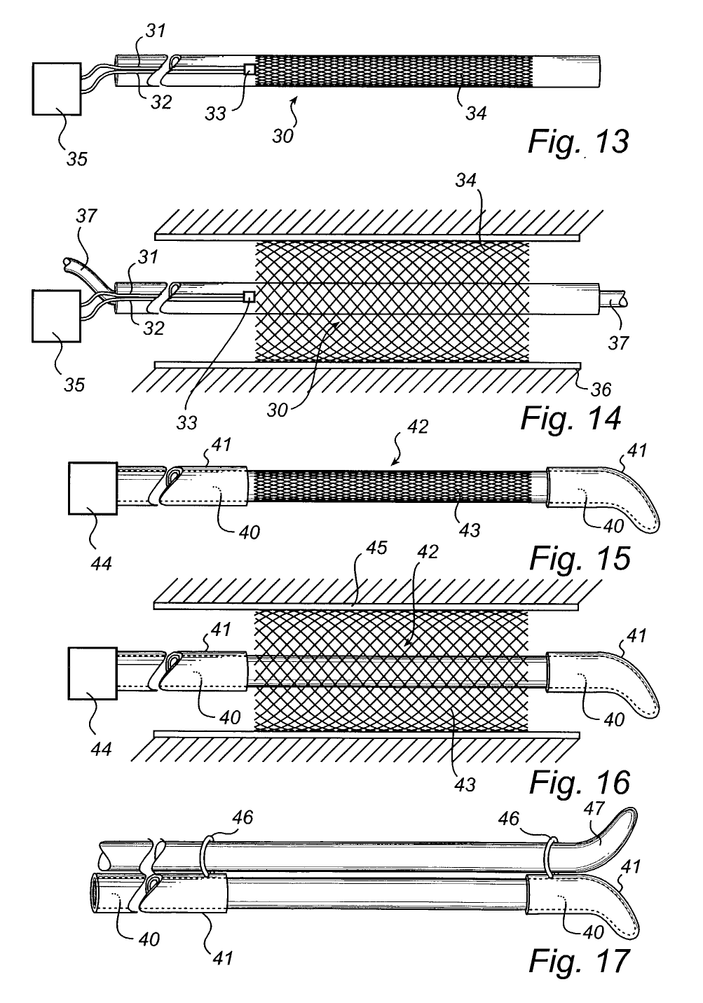

In the third embodiment shown in FIGS. 13-14, a catheter 30 has tubes 31, 32 coupled to a diminutive cooling head 33 which is connected to a stent 34 at a single point (point contact) or along a row of points (line contact). This embodiment takes advantage of the good heat conductivity of the metal stent 34. Thus, there is no need for the cooling cavities in the distal part of the catheter 30 which can have a more simple design. FIG. 13 shows the tubes 31, 32 coupled to a cooling source 35 so that the stent 34 is in its contracted state. In FIG. 14, the catheter 30 has been introduced to a desired site in a body vessel 36 by means of a guide wire 37, or through another guiding catheter (not shown), and then the supply of the cooling medium has been interrupted. Consequently, the temperature of the stent 34 has increased such that it has dilated to its expanded state. Finally, the catheter 30 and the guide wire will be withdrawn from the body vessel 36.

The fourth embodiment shown in FIGS. 15-16 uses a metal positioning wire 40 with an enclosing isolation layer 41 along its length except for at a distal part 42 thereof. Here, the heat conductivity of the positioning wire 40 is such as to make it possible to keep the non-isolated distal part 42 below the transformation temperature, or as close as possible to the freezing point, by cooling the metal wire 40 at a proximal part thereof, preferably external to the body. A stent 43 is kept in its contracted state by the cooling of the metal wire 40 at its proximal end by a cooling source 44. This could be accomplished by the metal wire 40 comprising a material which is strongly heat-conducting. Thus, heat will be transferred from the stent 43 to the non-isolated distal part 42 through the metal wire 40 to its proximal part.

In FIG. 16, the metal positioning wire 40 is inserted into a body vessel 45 through a guiding catheter (not shown) such that the distal part 42 of the metal wire 40 is positioned at a desired site. Further, the cooling of the metal wire 40 has been shut off, whereby the stent 43 has dilated to its expanded state and makes contact with the inside of the body vessel 45. Finally, the metal positioning wire 40 is withdrawn from the body vessel 45. In FIGS. 15-17, the distal end of the positioning wire 40 is bent to illustrate that the positioning wire 40 may be pliable.

FIG. 17 illustrates a modified positioning wire 40 having eyelets 46 for receiving a guide wire 47. Then a very flexible guide wire 47 may first be introduced to the desired position where the self-expanding device is to be placed. Thereafter, the positioning wire 40 may be led by means of the eyelets 46 along the guide wire 47 to the desired position. The eyelet 46 may be hingedly connected to the positioning wire 40 so that it may easily follow bends of the guide wire 47. The eyelets may also be replaced by one or more tubes attached to the delivery wire, letting the guide wire 47 through.

A method for delivery of a self-expanding device comprises positioning the self-expanding device around a distal part of a delivery means. The self-expanding device could be positioned around the distal part of the catheter during production of the delivery means. The self-expanding device may then be kept in a contracted state by a restraining means until a cooling of the device is initiated when the device is to be delivered into the body. Alternatively, the self-expanding device may simply be attached and contracted by hand around the cold distal part of the delivery means, before the device is to be delivered into the body.

The self-expanding device is cooled when positioned around the distal part of the delivery means during introduction into the body. Thus, the self-expanding device is kept in a contracted state while being delivered by the delivery means to a desired position. When the self-expanding device is properly positioned, the cooling is suspended, whereby the self-expanding device is deployed by self-expansion.

It will be appreciated that a number of further modifications of the above-described embodiments of the delivery apparatus are possible within the scope of the invention, as specified in the appended claims. As an example, the stent may be fixed on the distal part of the catheter or on the delivery wire preparatory to the insertion into a body vessel. An expanded stent of room temperature may on the other hand be positioned on the cooled distal part of the catheter or delivery wire by the operator who may use his fingers to hold the stent in position until it is so cold as to remain on the distal part of the catheter or delivery wire.

As a further example, the cooling of the self-expanding device during the introduction may be accomplished by means of a thermoelectric element. In this embodiment, the thermoelectric element is arranged at the distal part of the elongate delivery means. The thermoelectric element is coupled to a circuit for supplying power to the thermoelectric element. The circuit may be accomplished by connecting the thermoelectric element to conductors along the elongate delivery means. The conductors are connected to a power source at a proximal part of the elongate delivery means. When an electric current is applied to the thermoelectric element, it will have a cooling effect on the self-expanding device. The cooling may be suspended by means of an electric switch arranged at the proximal part of the elongate delivery means.

1. An apparatus for delivery of a temperature-sensitive, self-expandable device into a body vessel, said apparatus comprising:
an elongate delivery means having a distal part and a proximal part, said delivery means being arranged for carrying the self-expandable device in a contracted state at said distal part to a desired position in the body vessel, said delivery means further comprising a cooling means having a cooling surface at said distal part and being arranged to transfer heat from said device via the cooling surface towards said proximal part for maintaining the self-expandable device in said contracted state at a temperature below a transformation temperature of the device.

2. The apparatus according to claim 1, wherein the cooling means is arranged to suspend said transfer of heat, whereby said device is allowed to self-expand to an expanded state.

3. The apparatus according to claim 1, wherein the cooling means is arranged to transfer heat during said carrying of the self-expanding device to said desired position.

4. The apparatus as claimed in claim 1, wherein said cooling means comprises a cooling medium for cooling said cooling surface and for providing said transfer of heat.

5. The apparatus as claimed in claim 4, wherein said cooling means is arranged to prevent said cooling medium from entering the body vessel at said distal part.

6. The apparatus as claimed in claim 1, further comprising a means for temporary warming of the device when introduced to said desired position.

7. The apparatus as claimed in claim 1, further comprising restraining means for keeping the self-expanding device in its contracted state on the distal part of the elongate delivery means before the transfer of heat by the cooling means.

8. The apparatus as claimed in claim 7, wherein the restraining means is detachable from the elongate delivery means.

9. The apparatus as claimed in claim 2, wherein the elongate delivery means is a catheter on which said distal part is provided for carrying said device.

10. The apparatus as claimed in claim 9, wherein the catheter comprises a guide wire tube for receiving a guide wire.

11. The apparatus as claimed in claim 4, wherein the cooling means comprises a cooling tube for delivery of said cooling medium.

12. The apparatus as claimed in claim 9 and 11, wherein the cooling tube is arranged in the catheter for delivery of the cooling medium to the distal part of the catheter.

13. The apparatus as claimed in claim 12, wherein the catheter comprises a draining tube for draining the cooling medium from the distal part thereof.

14. The apparatus as claimed in claim 12, further comprising a source of the cooling medium for coupling to the cooling tube at a proximal end of the catheter.

15. The apparatus as claimed in claim 9, wherein the distal part of the catheter is dilatable.

16. The apparatus as claimed in claim 15, wherein dilatation of the distal part of the catheter is initiated by the suspending of the cooling.

17. The apparatus as claimed in claim 9, wherein the catheter comprises a balloon near a distal end thereof.

18. The apparatus as claimed in claim 17, wherein the balloon is positioned on said distal part of the catheter.

19. The apparatus as claimed in claim 9, wherein the distal part of the catheter comprises at least one pad for said heat transfer from the self-expanding device to the cooling means.

20. The apparatus as claimed in claim 19, wherein the pad is in contact with the self-expanding device over a substantial portion thereof.

21. The apparatus as claimed in claim 19, wherein the pad is in contact with the self-expanding device along a line.

22. The apparatus as claimed in claim 19, wherein the pad is in point contact with the self-expanding device.

23. The apparatus as claimed in claim 1, wherein said cooling means comprises a solid means extending between said distal and proximal parts for providing said transfer of heat.

24. The apparatus as claimed in claim 23, wherein said solid means is of a metal material.

25. The apparatus as claimed in claim 24, wherein said solid means is a positioning wire.

26. The apparatus as claimed in claim 25, wherein the elongate delivery means is said positioning wire on which said distal part is provided.

27. The apparatus as claimed in claim 26, wherein the positioning wire is isolated except for the distal part thereof.

28. The apparatus as claimed in claim 26, wherein the cooling means is connected to a proximal part of the positioning wire.

29. The apparatus as claimed in claim 1, wherein the self-expanding device is a memory metal medical device.

30. The apparatus as claimed in claim 1, wherein the self-expanding device is a stent.

31. The apparatus as claimed in claim 2, wherein the self-expanding device comprises cavities containing a drug in a solid state when cooled during said carrying and transformed to a liquid state when the cooling is suspended.

32. The apparatus as claimed in claim 1, wherein the self-expanding device comprises a drug in capsules adhering to the self-expanding device when cooled during said carrying.

33. The apparatus as claimed in claim 18, wherein the balloon comprises cavities containing a drug in a solid state when cooled during said carrying and transformed to a liquid state when the cooling is suspended.

34. The apparatus as claimed in claim 18, wherein the balloon comprises a drug in capsules adhering to the balloon when cooled during said carrying.

35. An apparatus for delivery of a temperature-sensitive, self-expandable device into a body vessel, said apparatus comprising:
an elongate delivery means having a distal part and a proximal part, said delivery means being arranged for carrying the self-expandable device in a contracted state at said distal part to a desired position in the body vessel, said delivery means further comprising a cooling means comprising a thermoelectric element at the distal part of the delivery means and being arranged to transfer heat from said device for maintaining the self-expandable device in said non-expanded state at a temperature below a transformation temperature of the device.

36. A method for delivery of a self-expanding device into a body vessel, said device in a contracted state thereof having a smaller diameter than a diameter of the body vessel and being self-expanding to a diameter at least equal to the diameter of the body vessel when the temperature of the device exceeds a transformation temperature, which is lower than the body temperature, said method comprising:
positioning said device on a distal part of an elongate flexible delivery means,
cooling the self-expanding device when positioned on said distal part by transferring heat from the device towards a proximal part of the delivery means for maintaining the self-expanding device in its contracted state,
introducing said distal part of delivery means with the self-expanding device to a desired position in the body vessel, and
suspending or at least reducing the cooling of the self-expanding device such that the self-expanding device is allowed to self-expand to an expanded state.

37. The method as claimed in claim 36, further comprising the step of warming the self-expanding device when introduced to said desired position.

38. The method as claimed in claim 36, wherein said step of positioning comprises the step of keeping said device contracted on the distal part of the elongate delivery means until the step of cooling the device before the introduction of the delivery means into the body vessel.

39. The method as claimed in claim 38, wherein said step of keeping the device contracted is achieved by a mechanical restraining means which is detached before the introduction of the delivery means into the body vessel.

40. The method as claimed in claim 36, wherein a catheter is used as the elongate delivery means.

41. The method as claimed in claim 40, wherein the step of cooling is provided by a cooling medium.

42. The method as claimed in claim 41, wherein the step of cooling further comprises the step of delivering the cooling medium to the distal part of the catheter via a tube therein.

43. The method as claimed in claim 42, wherein the cooling medium is drained from the distal part of the catheter via a second tube therein.

44. The method as claimed in claim 36, wherein heat is transferred from the self-expanding device to the cooling medium via at least one pad.

45. The method as claimed in claim 36, wherein a positioning wire, which is isolated except for the distal part thereof, is used as the elongate delivery means.

46. The method as claimed in claim 45, wherein cooling a proximal part of the positioning wire provides the step of cooling.

47. The method as claimed in claim 36, wherein the self-expanding device is compressed for keeping it contracted on the distal part of the elongate delivery means before the cooling thereof.

48. The method as claimed in claim 36, wherein a stent is used as the self-expanding device.

49. A method for delivery of a self-expanding device into a body vessel, said device in a contracted state thereof having a smaller diameter than a diameter of the body vessel and being self-expanding to a diameter at least equal to the diameter of the body vessel when the temperature of the device exceeds a transformation temperature, which is lower than the body temperature, said method comprising:
positioning said device on a distal part of an elongate flexible delivery means,
cooling the self-expanding device when positioned on said distal part by a thermoelectric element at said distal part of the delivery means for maintaining the self-expanding device in its contracted state,
introducing said distal part of delivery means with the self-expanding device to a desired position in the body vessel, and
suspending or at least reducing the cooling of the self-expanding device such that the self-expanding device is allowed to self-expand to an expanded state.

50. A method for controlling expansion of a self-expandable device to be located at a position in a body vessel by the use of an elongate insertion instrument, comprising the step of using said insertion instrument for transferring heat from the device, during introduction of the device to said position, in order to prevent an expansion of the device.
